(19) United States
(12) Patent Application Publication
(10) Pub. No.: US 2024/0167068 A1
(43) Pub. Date: May 23, 2024

(54) METHOD FOR RNA MANUFACTURING

(71) Applicant: eTheRNA immunotherapies NV, Niel (BE)

(72) Inventors: Senne DILLEN, Niel (BE); Lore DE BRUYNE, Niel (BE); Michiel HOLTOF, Niel (BE)

(21) Appl. No.: 18/569,372

(22) PCT Filed: Jun. 30, 2022
(86) PCT No.: PCT/EP2022/068012

§ 371 Date: Dec. 12, 2023

(30) Foreign Application Priority Data
Jun. 30, 2021 (EP) 21182703.5

(51) Int. Cl. C12P 19/34; C12N 9/12; G01N 21/59; G01N 27/06

(52) U.S. Cl. CPC C12P 19/34; C12N 9/1252; G01N 21/59; G01N 27/06

(57) ABSTRACT

The present invention relates to the field of nucleic acid production, in particular in vitro RNA transcription. More specifically, the present invention relates to a method wherein physicochemical properties of the reaction mixture are monitored in order to timely stop the reaction and/or to add fresh reagents to allow the reaction to continue under optimal conditions. In particular, conductivity, but also pH and/or optical density is determined at at least two different time points, or continuously during the in vitro transcription reaction.

### FIELD OF THE INVENTION

The present invention relates to the field of nucleic acid production, in particular in vitro RNA transcription. More specifically, the present invention relates to a method wherein physicochemical properties of the reaction mixture are monitored in order to timely stop the reaction and/or to add fresh reagents to allow the reaction to continue under optimal conditions. In particular, conductivity, but also pH and/or optical density is determined at at least two different time points, or continuously during the in vitro transcription reaction.

### BACKGROUND TO THE INVENTION

Production of mRNA drug substances is generally performed using batch-based methods. In such methods, an in vitro transcription (IVT) reaction is performed wherein reagents are added to the reaction vessel before the onset of the incubation period. During the incubation period, reagents (most notably NTPs, magnesium, and capping molecules) are depleted and/or the buffer capacity may change, after which the accumulation of mRNA within the reaction mix ceases. At this point, the reaction is typically terminated and the produced mRNA is isolated from the reaction mix using a variety of downstream processes. In such a batch-based production process, reagents that are essential to the performance of IVT but that are not consumed during the reaction (e.g. enzymes, DNA template) are lost at the end of the incubation period.

In a non-batch based manufacturing process, consumed reagents are freshly added to the reaction vessel as RNA accumulation ceases, thereby allowing synthesis of mRNA to continue beyond the point possible in a standard batch-based process, thereby transitioning from a batch-based to a (semi-)continuous manufacturing process.

Furthermore, also for batch manufacturing it is important to correctly time the moment upon which the reaction is actually stopped. An inefficient reaction due to depletion of reagents may for example result in accumulation of unwanted by-products such as double stranded RNA (dsRNA) or truncated RNA fragments.

The timing of ending a reaction or further addition of the required reagents can be based on incubation time (e.g. 5 ml of NTP solution is added after 2 h of incubation). Reagent consumption is dependent on many different factors though. A small change in salt content of the IVT reaction may for example increase or decrease the efficiency of transcription and thereby affect reagent-depletion rate or by-product formation. Furthermore, it is to be expected that certain RNA constructs will be transcribed more easily than others, causing reagent-depletion rate to be specific to each RNA construct. This means that thorough validation of each RNA construct-reagent mix combination would be needed if a reaction is to be stopped or reagents are to be added based on time alone.

It was therefore an object of the present invention to provide a solution to this problem, in particular to better time the ending of the reaction or addition of fresh reagents to the reaction. We have now found that the present problem may be solved by determining specific physicochemical characteristics of the IVT reaction.

The present invention thus relates to a method wherein physicochemical properties are monitored and the reaction is timely stopped, or fresh reagents are added in a timely manner to allow the reaction to continue under optimal conditions. In particular, the inefficient phase of RNA synthesis in the IVT reaction can be detected by monitoring the conductivity, optical density (OD), and/or pH of the reaction mix. Changes to these measurable parameters can then be used as a signal to trigger the ending of the reaction or addition of reagents, eliminating the need for extensive determination of the reagent-depletion rates for each RNA construct-reaction mix combination.

Such a method or manufacturing system allows the generation of a large amount of RNA from a relatively small volume of IVT reaction mix, resulting in a significantly reduced device footprint. Another advantage is that the reaction can timely be stopped or continue in optimal conditions in order to prevent the accumulation of dsRNA, a difficult to remove product-related impurity of in vitro transcribed RNA. Lastly, cost-of-goods is significantly reduced as less consumables and raw materials are consumed.

### SUMMARY OF THE INVENTION

In a first aspect, the present invention provides a method for manufacturing RNA using an in vitro transcription (IVT) reaction comprising determining the conductivity during said IVT reaction at at least two different time points, wherein upon detecting a decrease in conductivity between said at least two time points, the IVT reaction is stopped or supplemented with fresh reagents.

In a further embodiment, said conductivity is determined continuously during the reaction, and wherein upon detecting a decreasing trend in conductivity, the reaction is stopped or supplemented with fresh reagents.

In a particular embodiment, wherein upon detecting a decrease in conductivity of at least about 2% between said at least two time points, the reaction is stopped or supplemented with fresh reagents.

In yet a further embodiment, the method further comprises determining the pH and/or optical density (OD) at said at least two different time points during said transcription reaction.

In a further embodiment, wherein said pH and/or optical density is determined continuously during the reaction, and wherein upon detecting a decreasing trend in conductivity, in combination with a stabilizing trend in the pH and/or an increasing trend in the optical density, the reaction is stopped or supplemented with fresh reagents.

In yet a further embodiment, wherein upon detecting a decrease in conductivity in said reaction, in combination with a stabilization in the pH and/or an increase in the OD of said reaction between said at least two time points, the reaction is stopped or supplemented with fresh reagents.

In a specific embodiment, said increase in OD is at least about 2%.

In yet a further embodiment, said RNA manufacturing is performed in a batch, continuous or semi-continuous process.

In a further embodiment, said fresh reagents are selected from the list comprising: NTPs, capping molecules, enzyme cofactors, acids, bases, and/or pH buffer agents.

In a further aspect, the present invention provides the use of conductivity measurements during an in vitro transcription reaction in the manufacturing of RNA.

### BRIEF DESCRIPTION OF THE DRAWINGS

With specific reference now to the figures, it is stressed that the particulars shown are by way of example and for purposes of illustrative discussion of the different embodiments of the present invention only. They are presented in the cause of providing what is believed to be the most useful and readily description of the principles and conceptual aspects of the invention. In this regard no attempt is made to show structural details of the invention in more detail than is necessary for a fundamental understanding of the invention. The description taken with the drawings making apparent to those skilled in the art how the several forms of the invention may be embodied in practice.

FIG. 1: RNA content and relative double stranded RNA (dsRNA) content of the IVT reaction mix over time.

The RNA content (μg/ml) in the IVT mix rises steadily as incubation time increases but RNA synthesis ceased or is reduced at ±165 minutes of incubation (panel A).

The end of RNA synthesis coincides with a marked increase in the relative abundance of dsRNA (μg/mg RNA) (a major product-related impurity of RNA synthetized through in vitro transcription), also occurring at ±165 minutes of incubation and onwards (panel B).

FIG. 2: Physicochemical properties of the IVT reaction mix over time.

Panel A, conductivity (mS/cm): the steadily increasing conductivity signal shows a sharp and sudden decrease when the majority of dsRNA is formed (at approximately 165 minutes, see arrow, and onwards).

Panel B, pH: The pH decreases during the incubation period but stabilizes at the point when RNA accumulation stops and dsRNA content increases (at approximately 165 minutes, see arrow, and onwards).

Panel C, optical density (ΔOD (AU)): Relative optic density of the IVT reaction mix determined over the full spectrum ranging from 200 to 650 nm. Values are expressed as difference compared to the level at t=0 minutes. The absorbance at lower wavelengths (<300 nm, light grey) decreases as RNA accumulates but at a certain point (±165 to ±180 minutes, see arrow, time of dsRNA accumulation), optic density shows an increased peak before transitioning back to a baseline level.

FIG. 3: Physicochemical properties of an IVT reaction mix over time.

Panel A, conductivity (mS/cm) of the IVT reaction mix over time.

Panel B, Relative optical density (ΔOD (AU)) of the IVT reaction mix at different wavelengths. Values are expressed as difference compared to the level at t=0 minutes.

Panel C, pH of the IVT reaction mix over time.

Panel D, RNA content of the IVT reaction mix over time.

FIG. 4: Physicochemical properties of an IVT reaction mix over time, wherein additional NTPs were added at 240 minutes*.

Panel A, conductivity (mS/cm): Conductivity stabilized at ±185 minutes and started to decline at ±195 minutes. Conductivity once again started to rise after addition of NTP.

Panel B, optical density (ΔOD (AU)): Values are expressed as difference compared to the level at t=0 minutes. The optic density (graph shown for OD at 260 nm) declines as the reaction progressed. At 225 minutes, a sharp transient peak in OD once again occurred.

Panel C, RNA content: The termination of RNA synthesis closely correlates with the decline in conductivity observed in panel A and the transient peak in OD (260 nm) in panel B.

*The arrow indicates the moment of NTP addition.

### DETAILED DESCRIPTION OF THE INVENTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

As used in the specification and the appended claims, the singular forms “a”, “an”, and “the” include plural referents unless the context clearly dictates otherwise. By way of example, “a compound” means one compound or more than one compound.

The term “about” or “approximately” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

The present invention relates to a method wherein physicochemical properties are monitored and a reaction is stopped or fresh reagents are added in a timely manner to allow the reaction to continue under optimal conditions. The inventors of the present invention have found that accumulation of mRNA can be monitored with easily measurable parameters such as conductivity as well as pH and optical density (OD). Based on this information, the main advantage is to either add additional reagents to prevent the reaction from running in suboptimal conditions or to terminate the reaction before the bulk of product-related impurities (dsRNA) are formed. In addition, the described method is compatible with a (semi-)continuous RNA manufacturing process wherein the in vitro transcribed mRNA can be extracted from the IVT reaction, preventing the buildup of mRNA which would lead to increased viscosity and might negatively affect reaction conditions.

In a first aspect, the present invention provides a method for manufacturing RNA using an in vitro transcription (IVT) reaction comprising determining the conductivity during said IVT reaction at at least two different time points, wherein upon detecting a decrease in conductivity between said at least two time points, the IVT reaction is stopped or supplemented with fresh reagents.

In the context of the present invention, the term “RNA” relates to a molecule which comprises ribonucleotide residues and preferably being entirely or substantially composed of ribonucleotide residues. “Ribonucleotide” relates to a nucleotide with a hydroxyl group at the 2′-position of a β-D-ribofuranosyl group. In particular, the term refers to single stranded RNA, but may also refer to double stranded RNA, isolated RNA such as partially purified RNA, essentially pure RNA, synthetic RNA, recombinantly produced RNA, as well as modified RNA that differs from naturally occurring RNA by the addition, deletion, substitution and/or alteration of one or more nucleotides. Such alterations can include addition of non-nucleotide material, such as to the end(s) of a RNA or internally, for example at one or more nucleotides of the RNA. Nucleotides in RNA molecules can also comprise non-standard nucleotides, such as nucleotides non-naturally occurring or chemically synthesized nucleotides or deoxynucleotides. These altered RNAs can be referred to as analogs or analogs of naturally-occurring RNA.

According to the present invention, the term “RNA” includes and preferably relates to “mRNA” which means “messenger RNA” and relates to a “transcript” which may be produced using DNA as template and encodes a peptide or protein. mRNA typically comprises a 5′ untranslated region (5′-UTR), a protein or peptide coding region and a 3′ untranslated region (3′-UTR). mRNA has a limited halftime in cells and in vitro.

The term ‘modified mRNA molecules’ means mRNA molecules that contain one or more modified nucleosides (termed “modified nucleic acids”), which have useful properties such as the lack of a substantial induction of the innate immune response of a cell into which the mRNA is introduced. These modified nucleic acids enhance the efficiency of protein production, intracellular retention of nucleic acids, and viability of contacted cells, as well as possess reduced immunogenicity. An exemplary suitable modified nucleoside may for example by N1-methyl pseudouridine.

For the sake of clarity, a mRNA encompasses any coding RNA molecule, which may be translated by an eukaryotic host into a protein.

Preferably, mRNA is produced by in vitro transcription using a DNA template. In one embodiment of the invention, the RNA is obtained by in vitro transcription. The in vitro transcription methodology is known to the skilled person and may comprise a purified linear DNA template containing a promoter, ribonucleotide triphosphates, a buffer system that includes dithiothreitol (DTT) and magnesium ions, spermidine and an appropriate RNA polymerase such as T7 RNA polymerase. The exact conditions used in the transcription reaction depend on the amount of RNA needed for a specific application. There is a variety of in vitro transcription kits commercially available.

In the context of the present invention, the term “double stranded RNA” or “dsRNA” is meant to be any RNA molecule with sufficient internal homology to form significant secondary structures such as hairpins due to hybridization of internal complementary sequences with one another via Watson-Crick base pairing of nucleotide bases within the complementary sequences. Significant secondary structures generally involve stretches of homology greater than approximately nine bases, but the exact length depends to some extent on context and on whether such secondary structures impart any biological function to the molecule. In particular, molecules obtained after in vitro transcription typically comprise dsRNA with two separate complementary strands and may vary in size for example from 20 nucleotides to 200 nucleotides or even more than 500 nucleotides.

In the context of the present invention, dsRNA is formed as a byproduct identified in IVT reactions which can arise from T7 RNA-dependent RNA polymerase activity. In particular, three main types of byproduct in the IVT reaction may result in formation of dsRNA molecules. The first is formed by 3′-extension of the run-off products annealing to complementary sequences in the body of the run-off transcript either in cis (by folding back on the same RNA molecule) or trans (annealing to a second RNA molecule) to form extended duplexes. The second type of dsRNA molecules is formed by hybridization of an antisense RNA molecule to the run-off transcript. The antisense RNA molecules have been reported to be formed in a promoter- and run-off transcript-independent manner. Alternatively, a promoter-independent transcription of full-length anti-sense RNA has been also reported as a novel mechanism of dsRNA generation in T7 RNAPol-driven IVT reaction. A third form of dsRNA results from random pairing of abortive transcripts, either in cis (i.e. within the same molecule) or in trans (between two different molecules). According to the invention, dsRNA encompasses any kind of the described RNA byproducts in an IVT reaction.

In the context of the present invention, the terms ‘decrease’ or alternatively ‘decreasing’ are meant to be to a ‘reduction’, a ‘decline’, or a ‘drop’ in the physicochemical property measured during the IVT reaction. Accordingly, in a reaction where for example the conductivity would rise throughout the reaction under normal circumstances, the term ‘decrease’ means that said conductivity is lower then the conductivity measured at a previous time point in the IVT reaction. In particular, the physicochemical property such as conductivity, pH, and OD is preferably decreased by at least 2%, such as at least 3%, at least 4%, at least 5%, at least 6%, at least 7%, at least 8%, at least 9%, at least 10%, at least 20%, at least 30%, at least 40%, at least 50% when compared to the physicochemical property measures at a previous time point.

In the context of the present invention, the terms ‘increase or alternatively ‘increasing’ are meant to be to an ‘elevation’, an ‘increment’, or an ‘augmentation’ in the physicochemical property measured during the IVT reaction. Accordingly, in a reaction where for example the OD would decrease throughout the reaction under normal circumstances, the term ‘increase’ means that said OD is higher then the OD measured at a previous time point in the IVT reaction. In particular, the physicochemical property such as conductivity, pH, and OD is preferably increased by. at least 2%, such as at least 3%, at least 4%, at least 5%, at least 6%, at least 7%, at least 8%, at least 9%, at least 10%, at least 20%, at least 30%, at least 40%, at least 50% when compared to the physicochemical property measures at a previous time point.

As used herein, easily measurable physicochemical properties of the IVT reaction are used to time the addition of reagents or the stopping of the reaction. Determining physicochemical properties allows the IVT reaction to be monitored in real-time and reagents to be added when most appropriate, or the reaction to be stopped. Generally, the duration of an IVT reaction is 2-3 hours. However, at a certain point, one (or more) crucial reagents will become scarce and the IVT reaction will slow down and eventually come to a stop. Furthermore, reaction byproducts such as double stranded RNA may accumulate.

In the context of the present invention, the term ‘physicochemical properties’ is to be understood as measurable parameters such as conductivity, OD, and/or pH.

In the context of the present invention, physicochemical properties may be determined at at least two different time points i.e. the IVT reaction is not continuously monitored while the reaction is running. Said time points at which physicochemical properties can be determined can be throughout the entire IVT reaction but preferably after the reaction has progressed for some time sufficient to allow the reaction to initiate properly. In particular, and as evident from the examples part, at the start of the reaction, the physicochemical properties of the reaction may change drastically. However, after a short incubation period (typically about 20-30 min), this is normalized. Accordingly, the physicochemical properties of the reaction mixture are measured after about 5 min, such as about 10 min, about 15 min, about 20 min, about 30 min, about 40 min, about 50 min, about 60 min after starting the reaction.

In particular, during the incubation period, reagents (most notably NTPs, enzyme cofactors such as magnesium, capping molecules, acids, bases, and/or pH buffer agents) start to be depleted and/or the buffer capacity may change, depending of the used concentrations of reagents.

In a particular embodiment, ‘fresh reagents’ that are added to said IVT reaction are selected from the list comprising: NTPs, capping molecules, enzyme cofactors, acids, bases, and/or pH buffer agents.

In the context of the present invention, the term ‘capping molecules’ is to be understood as reagents necessary to product an RNA molecule of which the 5′ end is linked to a guanosine or a modified guanosine, preferably a 7-methylguanosine (N7-methyl guanosine or m7G), connected to a 5′ to 5′ triphosphate linkage or analogue. Capping molecules may include m7G, m7G carrying a methylated hydroxide group on any of the ribose sugars, NAD, NADH or 3′-dephospho-coenzyme A.

In the context of the present invention, the term ‘enzyme cofactors” is to be understood as a non-protein chemical compound or metallic ion that is required for an enzyme's activity as a catalyst (a catalyst is a substance that increases the rate of a chemical reaction). Enzyme cofactors may include ions, such as magnesium, zinc and iron ions, or organic molecules, such as vitamins or vitamin-derived molecules.

In the context of the present invention, the term ‘acids’ is to be understood as a molecule or ion capable of either donating a protein (i.e. hydrogen ion, H⁺) known as a Bronsted-Lowry acid, or forming a covalent bond with an electron pair, known as a Lewis acid. Common aqueous acids include HCl (hydrochloric acid), acetic acid (CH₃COOH), sulfuric acid (H₂SO₄) and citric acid.

In the context of the present invention, the term ‘bases’ is understood as a substance which dissociates in aqueous solution to form hydroxide ions (OH). Typical bases include lithium hydroxide (LiOH), sodium hydroxide (NaOH), potassium hydroxide (KOH), magnesium hydroxide (Mg(OH)₂) and calcium hydroxide (Ca(OH)₂)

In the context of the present invention, physicochemical properties may be determined at at least two different time points such as at least 3, at least 4, at least 5, at least 6, at least 7, at least 8, at least 9, at least 10, at least 20, at least 30, at least 40, at least 50 time points.

A series of measurements is set up to determine when a physicochemical property of a reaction changes from a steady increase to a steady decrease or from a steady decrease to a steady increase. Multiple measurements shortly after one another increase the reliability of the measurements, since small fluctuations in the measures can be corrected for. For example, only when multiple measures of the physicochemical properties of the reaction point in the same direction, i.e. change in increase/decrease status, appropriate responses are made. A single increased or decreased measure within a series of decreasing or increasing measures should not be a trigger for an appropriate response, e.g. addition of reagents or stopping of the reaction.

In a further embodiment, said physicochemical properties are determined continuously during the reaction.

For monitoring the reaction, not only singly or continuously measured properties at specific time points may be used, however, also the slope of the curve which connects the different measures in a graph can be used. Whenever there are significant changes in the slope, again an appropriate response can be made.

In a further embodiment, said conductivity is determined continuously during the reaction, and wherein upon detecting a decreasing trend in conductivity, the reaction is stopped or supplemented with fresh reagents.

In the context of the present invention, the term “conductivity” is to be understood as a measure of a material's ability to conduct an electric current, in particular it quantifies how well a material conducts electricity. The SI unit of conductivity is Siemens per meter (S/m). Conductivity measurements are used routinely in many applications as a fast, inexpensive and reliable way of measuring the ionic content in a solution. For example, the measurement of product conductivity is a typical way to monitor and continuously trend the performance of water purification systems. Nucleic acids such as DNA and RNA have shown to possess a small semi-conductivity in the dry state which is related to the high content of double-helix structure.

In a particular embodiment, wherein upon detecting a decrease in conductivity of at least about 2% between said at least two time points, the reaction is stopped or supplemented with fresh reagents.

In yet a further embodiment, the method further comprises determining the pH and/or optical density (OD) at said at least two different time points during said transcription reaction.

In a further embodiment, wherein said pH and/or optical density is determined continuously during the reaction, and wherein upon detecting a decreasing trend in conductivity, in combination with a stabilizing trend in the pH and/or an increasing trend in the OD, the reaction is stopped or supplemented with fresh reagents.

In yet a further embodiment, wherein upon detecting a decrease in conductivity in said reaction, in combination with a stabilization in the pH and/or an increase in the OD of said reaction between said at least two time points, the reaction is stopped or supplemented with fresh reagents.

In a particular embodiment, said increase in OD is at least about 2%.

In another particular embodiment, a stabilization in pH values may determine the point of ending the reaction and/or supplementing it with fresh reagents. In the context of the invention, the term ‘stabilization’ is meant to be the phase of the reaction wherein the pH difference between 2 or more consecutive measurements is small, such as at most 10% difference, in particular less than about 9%, 8%, 7%, 6%, 5%, 4%, 3%, 2%, or 1% difference between such consecutive measurements. Where the data are monitored on a graph, a small slope of the curve connecting 2 or more consecutive measurements, is also an indication of the stabilization in pH values. For example, the slope of the curve may be less then about 10%, in particular less than about 9%, 1-8%, 7%, 6%, 5%, 4%, 3%, 2%, or 1%.

In yet a further embodiment, said RNA manufacturing is performed in a batch, continuous or semi-continuous process. The method of the present invention is particularly suitable for continuous or semi-continuous process in which appropriate timing of the addition of fresh reagents is needed. Alternatively the method of the present invention may also be suitably used for batch processes to appropriately time the ending of the reaction, in order to reduce the formation of unwanted by-products (e.g. dsRNA), which specifically occurs towards the less efficient phase of the reaction.

A major advantage of the present invention is that expensive reagents such as DNA template, polymerases, enzymes, . . . can be used for a longer period of time, thereby significantly reducing the cost of RNA production. Specifically, these components are not consumed during the reaction, and by timing the addition of further reagents, larger batches of RNA can be prepared using a similar amount of starting reagents, and the addition of only the consumed reagents during the reaction.

In a further aspect, the present invention provides the use of conductivity measurements during an in vitro transcription reaction in the manufacturing of RNA.

### Examples

### Material and Methods

For the initial monitoring of the physicochemical properties (conductivity, pH, optical density) of the IVT reaction, 35 ml of caTLR4 IVT reaction mix was prepared and incubated at 37° C. for 4 hours. Every 15 minutes, the IVT reaction was sampled and optic density (200 to 650 nm), conductivity and pH of the reaction mix was determined. Samples were also subjected to LiCl precipitation in order to allow the determination of their RNA content. The relative abundance of dsRNA was determined using anti-dsRNA slot blot using the anti-dsRNA J2 antibody (Scicons).

### Results

### Experiment 1

RNA Content and dsRNA Content

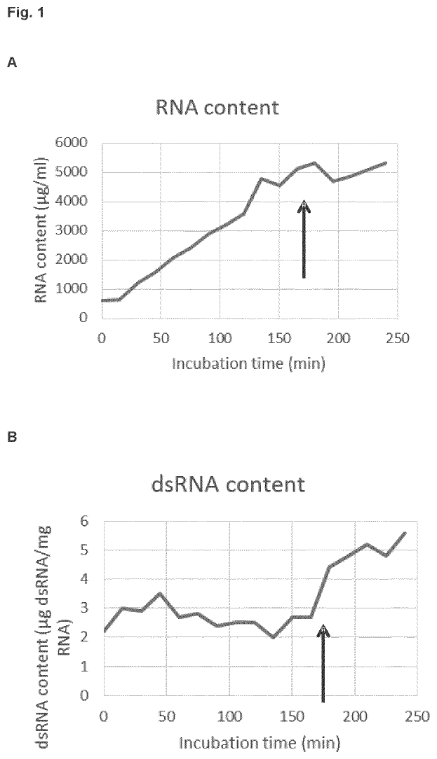

The RNA content of the samples rises steadily as incubation time increases. At ±165 minutes of incubation, however, the increase in RNA content ceases and the signal levels off, indicating that RNA synthesis has ceased at this point (FIG. 1A).

The end of RNA synthesis coincides with a marked increase in the relative abundance of dsRNA (a major product-related impurity of RNA synthetized through in vitro transcription), also occurring at ±165 minutes of incubation (FIG. 1B).

### Physicochemical Properties

After an initial reduction in conductivity, the conductivity signal steadily increases as the incubation period progresses. At 165 minutes, the signal stabilizes and starts a steep and rapid decline (FIG. 1A).

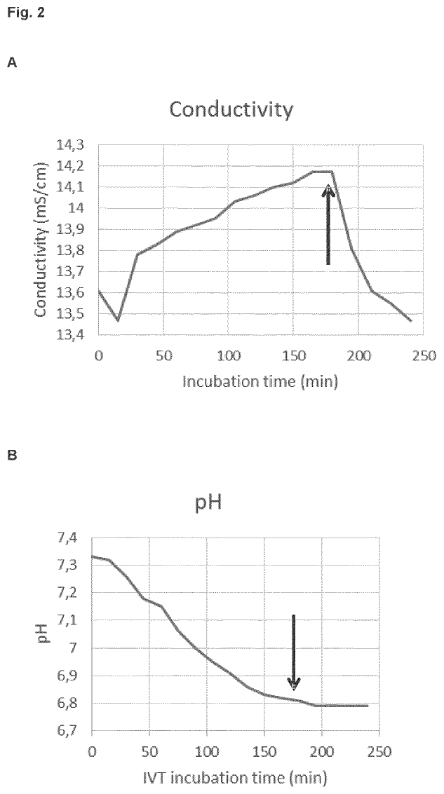

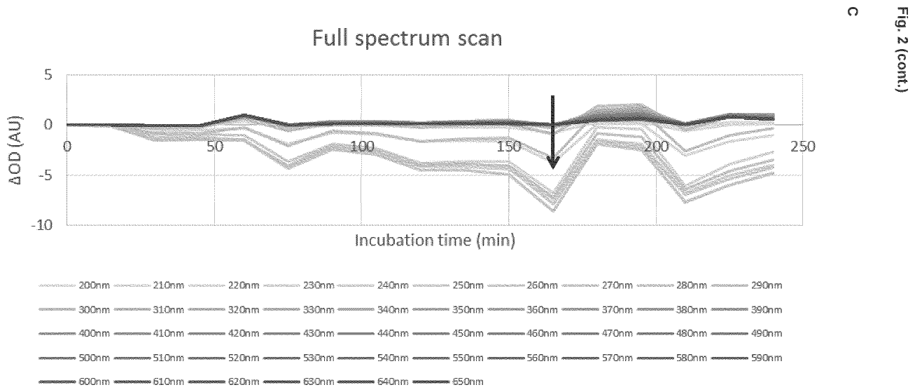

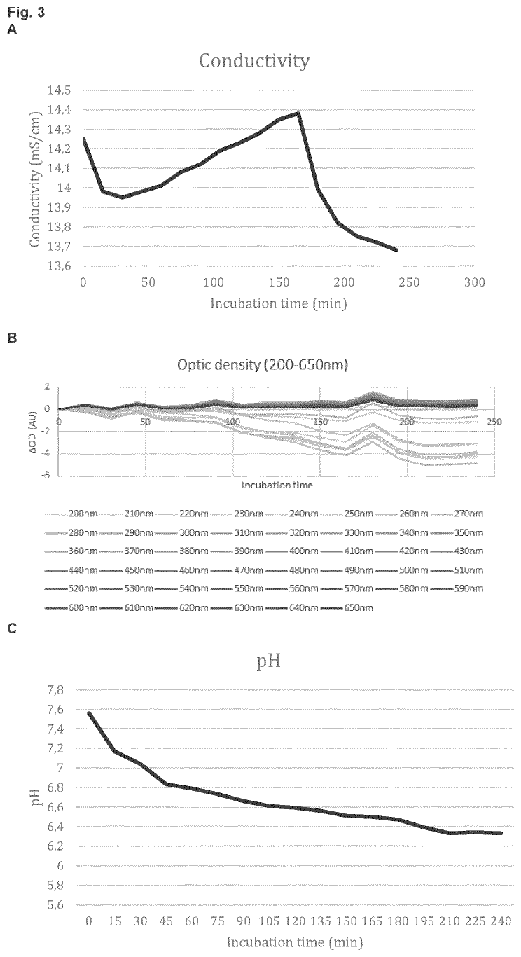

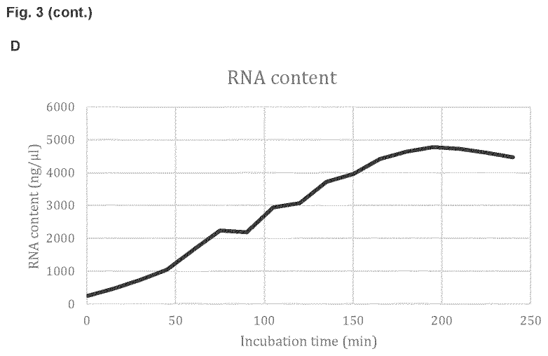

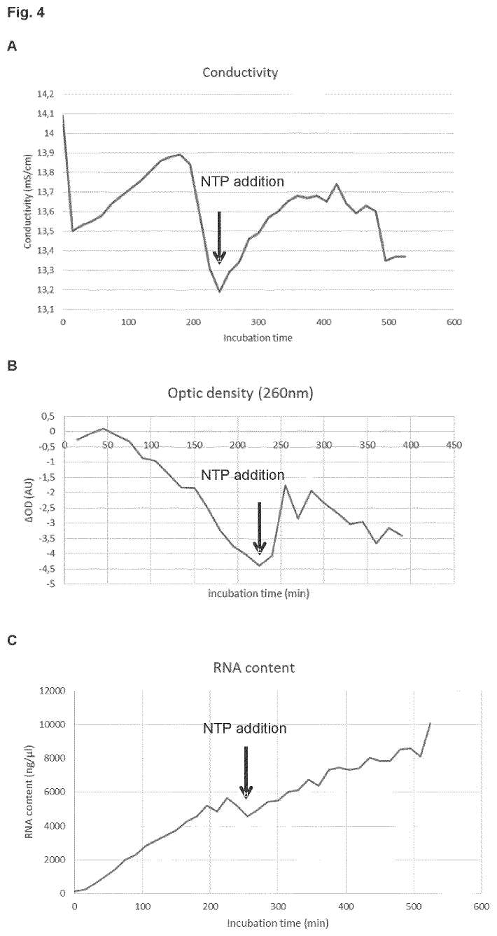

A significant increase in acidity can be observed as the reaction progresses. The reduction in pH can be seen to flatten off once the synthesis of mRNA ceases (165 minutes and onwards, FIG. 2B).

The optic density at lower wavelength (200-300 nm) slowly decreases as the reaction progresses, possibly indicating the decreasing concentration of NTPs in the reaction mixture.

At a certain point (±165 to ±180 minutes), however, optic density shows a small but remarkable peak before transitioning back to a baseline level (FIG. 1C).

### Experiment 2 (Confirmatory Study)

In order to confirm the results previously obtained in experiment 1, a confirmatory study was performed. This study was designed as an exact replicate of the previously performed experiments. As such, 35 ml of caTLR4 IVT mix was prepared and incubated for 4 hours. The reaction mix was sampled every 15 minutes and the conductivity, pH, optic density and RNA content of these samples was analyzed.

As was previously observed, the conductivity of the IVT reaction initially declined, after which it showed a steady increase as incubation time progressed. At ±165 minutes, a sharp decrease in conductivity was observed (FIG. 3A).

When the optic density of the reaction mix was monitored over time, this study confirms the results obtained in the previous experiment. While OD at higher wavelengths remains fairly stable, a notable decrease in OD is observed at shorter wavelengths (FIG. 3B). A transient peak in OD is observed from ±165 to ±195 minutes.

As previously observed, the pH of the reaction mix declined as the reaction progressed. At ±200 minutes, the signal stabilized, indicating that the release of protons into the reaction mix caused by the incorporation of NTPs in the RNA strand had ceased (FIG. 3C).

When the obtained data are correlated to the RNA content shown in FIG. 3D, the stabilization of the pH signal and the sharp decrease in conductivity once again coincide with the point at which the accumulation of RNA in the reaction mix ceases. At these same timepoints, a transient peak in optic density is also observed.

### Conclusion EXP. 1 and EXP. 2

The present experiment demonstrates that measurable differences in conductivity, pH, and OD coincide with the end of the accumulation of RNA in the reaction mix and the increase of the abundance of dsRNA.

### Experiment 3 (Proof of Principle)

A proof-of-principle experiment was performed in which 25 ml caTLR4 IVT mix was prepared and incubated at 37° C. Every 15 minutes, the reaction mix was sampled and pH, OD and conductivity were measured. Based on the observation described below, NTPs were added when an increase in OD and a sudden decrease in conductivity were observed. The addition of NTPs to the reaction mix was performed at 240 minutes.

As previously observed, the conductivity of the reaction mix initially showed a strong decline which was followed by a steady increase. Conductivity stabilized at ±185 minutes and started to decline at ±195 minutes. Whereas the conductivity signal remained stable when no additional NTPs were added (FIG. 3A), conductivity once again started to rise after addition of NTP, likely indicating that RNA synthesis had resumed. Conductivity once again stabilizes after ±360 minutes and starts to decline after ±420 (FIG. 4A).

As was also previously observed, the optic density (graph shown for OD at 260 nm) declines as the reaction progressed. At 225 minutes, however, a sharp transient peak in OD once again occurred (FIG. 4B).

In the previously described experiment 1, RNA accumulation had ceased after 165 minutes, flattening off at a level of ±5000 ng/μl. In this proof of principle experiment, however, the same upper plateau at 5000 ng/μl was observed, with the difference that this plateau value was only reached after ±210 minutes of incubation. The termination of RNA synthesis observed in FIG. 3C closely correlated with the decline in conductivity observed in FIG. 4A and the transient peak in OD (260 nm) observed in FIG. 4B.

Most notably, the timely addition of additional NTPs in the reaction mix caused a further increase of the RNA content of the reaction mix, which finally stabilized at a level of ±8500 ng/μl.

### Conclusion EXP. 3

The present experiment demonstrates that after adding fresh NTPs, the production of RNA is continued, exemplified by measurable differences in conductivity, pH, and OD.

1. A method for manufacturing RNA using an in vitro transcription reaction comprising determining the conductivity during said transcription reaction at at least two different time points, wherein upon detecting a decrease in conductivity between said at least two time points, the reaction is stopped or supplemented with fresh reagents.

2. The method according to claim 1, wherein said conductivity is determined continuously during the reaction, and wherein upon detecting a decreasing trend in conductivity, the reaction is stopped or supplemented with fresh reagents.

3. The method according to claim 1, wherein upon detecting a decrease in conductivity of at least about 2% between said at least two time points, the reaction is stopped or supplemented with fresh reagents.

4. The method according to anyone of claims 1 to 3, further comprising determining the pH and/or optical density (OD) at said at least two different time points during said transcription reaction.

5. The method according to claim 4, wherein said pH and/or optical density is determined continuously during the reaction, and wherein upon detecting a decreasing trend in conductivity, in combination with a stabilizing trend in the pH and/or an increasing trend in the optical density, the reaction is stopped or supplemented with fresh reagents.

6. The method according to claim 4, wherein upon detecting a decrease in conductivity in said reaction, in combination with a stabilization in the pH and/or an increase in the OD of said reaction between said at least two time points, the reaction is stopped or supplemented with fresh reagents.

7. The method according to claims 5 and 6, wherein said increase in OD is at least about 2%.

8. The method according to anyone of claims 1 to 7, wherein said RNA manufacturing is performed in a batch, continuous or semi-continuous process.

9. The method according to anyone of claims 1 to 8, wherein said fresh reagents are selected from the list comprising: NTPs, capping molecules, enzyme cofactors, acids, bases, and/or pH buffer agents.

10. Use of conductivity measurements during an in vitro transcription reaction in the manufacturing of RNA.
